(19) United States
(12) Patent Application Publication
(10) Pub. No.: US 2007/0098138 A1
(43) Pub. Date: May 3, 2007

(54) X-RAY DETECTOR AND X-RAY CT APPARATUS

(76) Inventor: Koji Bessho, Tokyo (JP)

Correspondence Address: Patrick W. Rasche;Armstrong Teasdale LLP, Suite 2600, One Metropolitan Square, St. Louis, MO, 63102, US

(21) Appl. No.: 11/554,820

(22) Filed: Oct. 31, 2006

(30) Foreign Application Priority Data
Nov. 1, 2005 (JP) 2005-318303

(51) Int. Cl. H05G 1/60; A61B 6/00; G01N 23/00; G21K 1/12

(52) U.S. Cl. 378/19

(57) ABSTRACT

To realize an X-ray detector and an X-ray CT apparatus with improved efficiency for X-ray utilization while leaving gaps existing among solid state detectors which are arranged two-dimensionally. A scintillator has a parallelepiped structure in which a top face and an under face are deviated from each other in a channel direction only by an amount d1 exceeding width of a gap, thereby eliminating an X-ray insensitive area when viewed from the X-ray incidence direction. Therefore, efficiency for X-ray utilization improves and, moreover, improvement in X-ray detectivity and picture quality of a slice image to be captured is realized.

### CROSS REFERENCE TO RELATED APPLICATIONS

This application claims the benefit of Japanese Application No. 2005-318303 filed Nov. 1, 2005.

### BACKGROUND OF THE INVENTION

The present invention relates to an X-ray detector and an X-ray CT apparatus each having solid state detectors which are repeatedly arranged two-dimensionally with gaps therebetween on a plane board on which X-rays are incident.

### BACKGROUND ART

In recent years, as X-ray detectors used for an X-ray CT apparatus, solid state detectors which are arranged two-dimensionally in a channel direction and a slice direction are used. The number of channels in the scan direction of the x-ray detectors and the number of X-ray detectors in the slice direction are increasing. For example, the number of X-ray detectors in the channel direction is about 1,000, and the number of X-rays in the slice direction is tens (refer to, for example, Japanese Patent Laid-Open No. 2004-093489 (p. 1 and FIG. 4).

Under the circumstances, the size of an X-ray receiving surface of a solid state detector is decreasing to a few mm². On the other hand, the width of each of gaps between solid state detectors, which are developed when the solid state detectors are arranged two-dimensionally is about 0.2 mm to 0.4 mm. The width of the gap is not largely changed with increase in the number of solid state detectors in the channel and slice directions but is constant more or less.

In the background art, however, efficiency for X-ray utilization of the solid state detectors arranged two-dimensionally deteriorates. Specifically, as the solid state detectors arranged two-dimensionally become finer, the proportion of the gaps increases as compared with the X-ray receiving surfaces of the solid state detectors, and the ratio of X-rays which pass without being detected by the solid state detectors increases.

In particular, the gaps of the solid state detectors are created in a process of producing a two-dimensional array of the solid state detectors and also play the role of preventing leakage (crosstalk) of fluorescence generated by X-rays among the solid state detectors. Therefore, it is not easy to reduce the size of the gap from the viewpoint of precision of a machine tool for processing the solid state detectors and performance of the solid state detectors.

It is consequently important to realize an X-ray detector and an X-ray CT apparatus with improved efficiency for X-ray utilization while leaving the gaps existing among the solid state detectors arranged two-dimensionally.

The present invention has been achieved to solve the problems of the background art and an object of the invention is to provide an X-ray detector and an X-ray CT apparatus with improved efficiency for X-ray utilization while leaving gaps existing among the solid state detectors which are arranged two-dimensionally.

### SUMMARY OF THE INVENTION

To solve the problems and achieve the object, the invention according to a first aspect provides an X-ray detector in which a plurality of solid state detectors each having a parallelepiped shape are arranged in a two-dimensional array with gaps therebetween on a plane board facing an X-ray incidence direction. Two parallel faces orthogonal to the incidence direction, of each of the parallelepipeds in the solid state detector have a positional deviation in the plane direction of the faces.

In the invention according to the first aspect, in the solid state detector, the gap portion is covered by the positional deviation in a plane direction between the two parallel faces which are facing the incidence direction.

According to the invention of a second aspect, in the X-ray detector according to the invention of the first aspect, the positional deviation is provided in at least one of a channel direction and a slice direction of the two-dimensional array.

In the invention according to the second aspect, the positional deviation exists in an arbitrary direction orthogonal to the X-ray incidence direction.

An X-ray detector according to the invention of a third aspect is characterized in that, in the invention of the first aspect, the positional deviation has a dimension exceeding width of the gap in the plane direction.

In the invention according to the third aspect, the plane board viewed from the X-ray incidence direction is covered with the solid state detectors.

An X-ray detector according to the invention of a fourth aspect is characterized in that, in the invention of the first aspect, the solid state detector is a scintillator.

In the invention according to the fourth aspect, the solid state detector detects an X-ray efficiently.

An X-ray detector according to the invention of a fifth aspect is characterized in that, in the invention of the fourth aspect, the plane board has a photodiode for detecting fluorescence generated by the scintillator.

In the invention according to the fifth aspect, the plane board efficiently converts fluorescence into an electric signal by the photodiode.

The invention according to a sixth aspect provides an X-ray CT apparatus having an X-ray detector in which a plurality of solid state detectors each having a parallelepiped shape are arranged in a two-dimensional array with gaps therebetween on a plane board facing an X-ray incidence direction, wherein a relative position in the X-ray incidence direction of two parallel faces of each of the parallelepipeds in the solid state detector have a positional deviation in the plane direction of the faces.

In the invention according to the sixth aspect, the solid state detectors cover gaps by the positional deviation in a plane direction between the two parallel faces which are facing the incidence direction.

An X-ray CT apparatus according to the invention of a seventh aspect is characterized in that, in the invention of the sixth aspect, the positional deviation has a dimension exceeding width of the gap in the plane direction.

In the invention according to the seventh aspect, the plane board viewed from the X-ray incidence direction is covered with the solid state detectors.

An X-ray CT apparatus according to the invention of an eighth aspect is characterized in that, in the invention of the sixth aspect, the solid state detector is a scintillator.

In the invention according to the eighth aspect, the solid state detector detects an X-ray efficiently.

An X-ray detector according to the invention of a ninth aspect is characterized in that, in the invention of the eighth aspect, the plane board has a photodiode for detecting fluorescence generated by the scintillator.

In the invention of the ninth aspect, the plane board efficiently converts fluorescence into an electric signal by the photodiode.

The invention of a tenth aspect provides an X-ray CT apparatus comprising: an X-ray tube that generates an X-ray; and an X-ray detector in which a plurality of solid state detectors each having a rectangular parallelepiped shape are arranged in a two-dimensional array with gaps therebetween on a plane board facing the X-ray incidence direction, wherein the plane board being tilted with respect to a direction orthogonal to the incidence direction.

In the invention according to the tenth aspect, the plane board has a tilt with respect to an X-ray incidence direction and the gaps between the solid state detectors are positioned in the shadow of the incident X-ray.

An X-ray CT apparatus according to the invention of an eleventh aspect is characterized in that, in the invention of the tenth aspect, the plane board being tilted so the X-ray projection of the rectangular parallelepiped as to exceed the gap and overlaps an adjacent rectangular parallelepiped.

In the invention according to the eleventh aspect, the tilt is set so that the projection of the rectangular parallelepiped covers the gap.

An X-ray CT apparatus according to the invention of a twelfth aspect is characterized in that, in the invention of the tenth aspect, the solid state detector is a scintillator.

In the invention according to the twelfth aspect, the solid state detector detects an X-ray efficiently.

An X-ray CT apparatus according to the invention of a thirteenth aspect is characterized in that, in the invention of the twelfth aspect, the plane board has a photodiode for detecting fluorescence generated by the scintillator.

In the invention according to the thirteenth aspect, the plane board efficiently converts fluorescence into an electric signal by the photodiode.

The invention according to a fourteenth aspect provides an X-ray detector in which a plurality of solid state detectors each having a rectangular parallelepiped shape are arranged in a two-dimensional array with gaps therebetween on a plane board facing an X-ray incident direction, wherein the X-ray detector has a multilayer solid state detector in which a plurality of the solid state detectors in the two-dimensional array are stacked in the incidence direction, and relative positions of the stacked solid-state detectors are deviated in direction orthogonal to the stacked direction.

In the invention according to the fourteenth aspect, the multilayer solid state detector has a configuration in which a plurality of two-dimensional arrays of the solid state detectors whose relative positions are deviated preferably only by the width of the gap are stacked in the incidence direction.

An X-ray detector according to the invention of a fifteenth aspect is characterized in that, in the invention of the fourteenth aspect, the two-dimensional array has the relative position which varies in at least one of a channel direction and a slice direction as two arrangement directions of the two-dimensional array.

In the invention of the fifteenth aspect, the relative positions of the two-dimensional arrays vary in an arbitrary direction orthogonal to the X-ray incidence direction.

An X-ray detector according to the invention of a sixteenth aspect is characterized in that, in the invention of the fifteenth aspect, the multilayer solid state detector has first, second, third, and fourth solid state detectors whose relative positions are different from each other.

In the invention according to the sixteenth aspect, the gaps viewed from the X-ray incidence direction are covered in the multilayer solid state detector.

An X-ray detector according to the invention of a seventeenth aspect is characterized in that, in the invention according to the fourteenth aspects, the solid state detector is a scintillator.

In the invention according to the seventeenth aspect, the solid state detector detects an X-ray efficiently.

The invention according to an eighteenth aspect provides an X-ray CT apparatus comprising an X-ray detector in which a plurality of solid state detectors each having a rectangular parallelepiped shape are arranged in a two-dimensional array with gaps therebetween on a plane board facing an incident X-ray, wherein the X-ray detector has a multilayer solid state detector in which a plurality of the solid state detectors in the two-dimensional array are stacked in the incidence direction, and relative positions of the stacked solid-state detectors are deviated in a direction orthogonal to the stacked direction.

In the invention according to the eighteenth aspect, the multilayer solid state detector has a configuration in which a plurality of two-dimensional arrays of solid state detectors whose relative positions are different preferably only by width of the gap are stacked in the incidence direction.

An X-ray detector according to the invention of a nineteenth aspect is characterized in that, in the invention of the eighteenth aspect, the relative position in the multilayer solid state detector varies in at least one of a channel direction and a slice direction as two arrangement directions of the two-dimensional array.

In the invention according to the nineteenth aspect, the relative position of the two-dimensional array varies in an arbitrary direction orthogonal to the X-ray incidence direction.

An X-ray detector according to the invention of a twentieth aspect is characterized in that, in the invention of the eighteenth aspect, the solid state detector is a scintillator.

In the invention according to the twentieth aspect, the solid state detector detects an X-ray efficiently.

According to the present invention, in the solid state detector, the gap portions are covered by the positional deviation in a plane direction of two parallel faces which are facing the incidence direction. Thus, the side on which an X-ray is incident of the plane board is covered with the solid state detectors, thereby eliminating an X-ray insensitive region. Thus, efficiency for X-ray utilization improves and, moreover, X-ray detectivity and the picture quality can be improved.

### BRIEF DESCRIPTION OF THE DRAWINGS

FIG. 1 is a block diagram showing a general configuration of an X-ray CT apparatus.

FIG. 2 is an outside diagram of an X-ray detector of a first embodiment.

FIG. 3 is an outside diagram of a plane block of the first embodiment.

FIG. 4 is a cross section of the plane block of the first embodiment.

FIGS. 5A and 5B are explanatory diagrams illustrating operations of the plane block of the first embodiment.

FIGS. 6A and 6B are an outside diagram and a cross section, respectively, of a plane block of a second embodiment.

FIG. 7 is an explanatory diagram showing operation of the plane block of the second embodiment.

FIGS. 8A and 8B are a cross section and an outside diagram, respectively, of a plane block of a third embodiment.

FIGS. 9A to 9D are plan views of multilayer scintillators constructing the plane block of the third embodiment.

FIG. 10 is an explanatory diagram showing operation of the plane block of the third embodiment.

### DETAILED DESCRIPTION OF THE INVENTION

Best modes for carrying out an X-ray detector and an X-ray CT apparatus according to the present invention will be described below with reference to the appended drawings. The present invention, however, is not limited to the best modes.

### First Embodiment

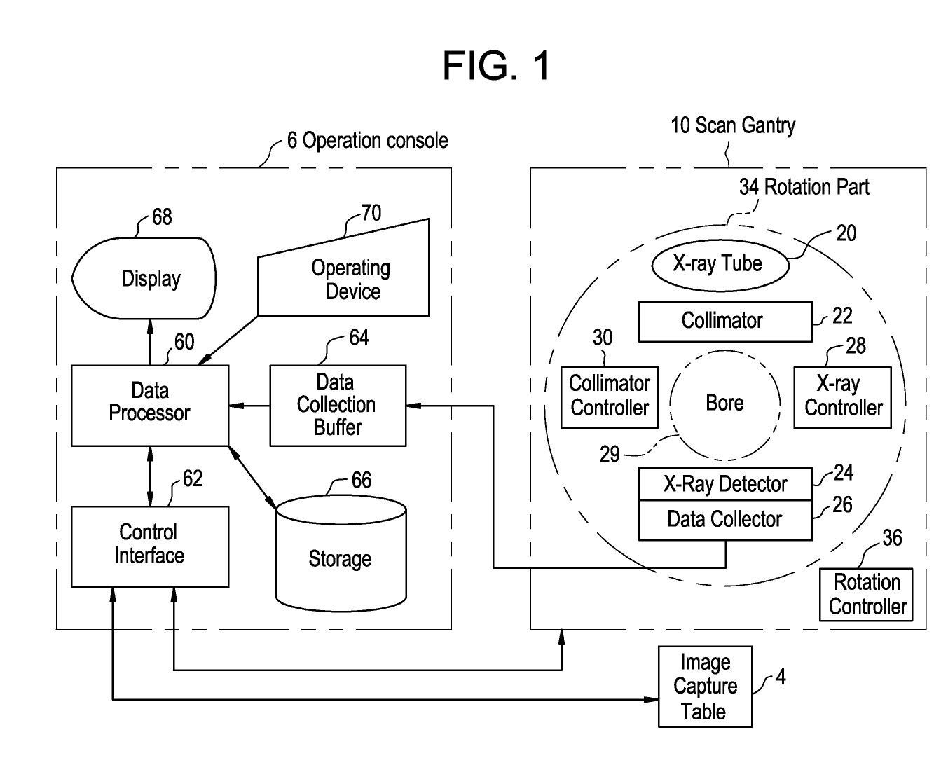

A general configuration of an X-ray CT apparatus according to a first embodiment will be described. FIG. 1 is a block diagram of an X-ray CT apparatus. As shown in FIG. 1, the apparatus has a scan gantry 10 and an operation console 6.

The scan gantry 10 has an X-ray tube 20. A not-shown X-ray emitted from the X-ray tube 20 spreads, for example, in a fan shape having a thickness and is shaped into a conical X-ray beam by a collimator 22 and is emitted to an X-ray detector 24.

The X-ray detector 24 has a plurality of scintillators arranged in a matrix in the spread direction of the fan-shaped X-ray beam. The X-ray detector 24 is a multi-channel detector having a width in which a plurality of scintillators are arranged two-dimensionally in a matrix in the channel direction and the slice direction.

In the X-ray detector 24, an X-ray incident surface curved in a concave shape as a whole is formed. The X-ray detector 24 is obtained by combining, for example, scintillators as solid state detectors made of inorganic crystal and a photodiode as a photoelectric converter.

To the X-ray detector 24, a data collector 26 is connected. The data collector 26 collects detection information of each of the scintillators of the X-ray detector 24. The irradiation of the X-ray from the X-ray tube 20 is controlled by an X-ray controller 28. The connection relation between the X-ray tube 20 and the X-ray controller 28 and the connection relation between the collimator 22 and a collimator controller 30 are not shown. The collimator 22 is controlled by the collimator controller 30.

The above-described components from the X-ray tube 20 to the collimator controller 30 are mounted on a rotation part 34 of the scan gantry 10. A subject or a phantom is placed on an image capture table 4 in a bore 29 positioned in the center of the rotation part 34. The rotation part 34 rotates while being controlled by a rotation controller 36, an X-ray is emitted from the X-ray tube 20, and the X-ray detector 24 detects the X-ray passed through the subject or phantom as projection information of each view according to the rotation angle. The connection relation between the rotation part 34 and the rotation controller 36 is not shown.

The operation console 6 has a data processor 60. The data processor 60 is constructed by, for example, a computer. To the data processor 60, a control interface 62 is connected. To the control interface 62, the scan gantry 10 is connected. The data processor 60 controls the scan gantry 10 via the control interface 62.

The data collector 26, X-ray controller 28, collimator controller 30, and rotation controller 36 in the scan gantry 10 are controlled via the control interface 62. The connection between each of those components and the control interface 62 is not shown here.

To the data processor 60, a data collection buffer 64 is connected. The data collection buffer 64 is connected to the data collector 26 in the scan gantry 10. Data collected by the data collector 26 is input to the data processor 60 via the data collection buffer 64.

The data processor 60 reconstructs an image by using a transmission X-ray signal, that is, projection information collected via the data collection buffer 64. To the data processor 60, a storage 66 is connected. The storage 66 stores the projection information collected by the data collection buffer 64, reconstructed slice image information, a program for realizing the functions of the apparatus, and the like.

To the data processor 60, a display 68 and an operating device 70 are connected. The display 68 displays the slice image information and other information which is output from the data processor 60. The operating device 70 is operated by an operator and inputs various instructions, information, and the like to the data processor 60. The operator operates the apparatus interactively by using the display 68 and the operating device 70. The scan gantry 10, the image capture table 4, and the operation console 6 radiograph the subject or phantom to obtain slice images.

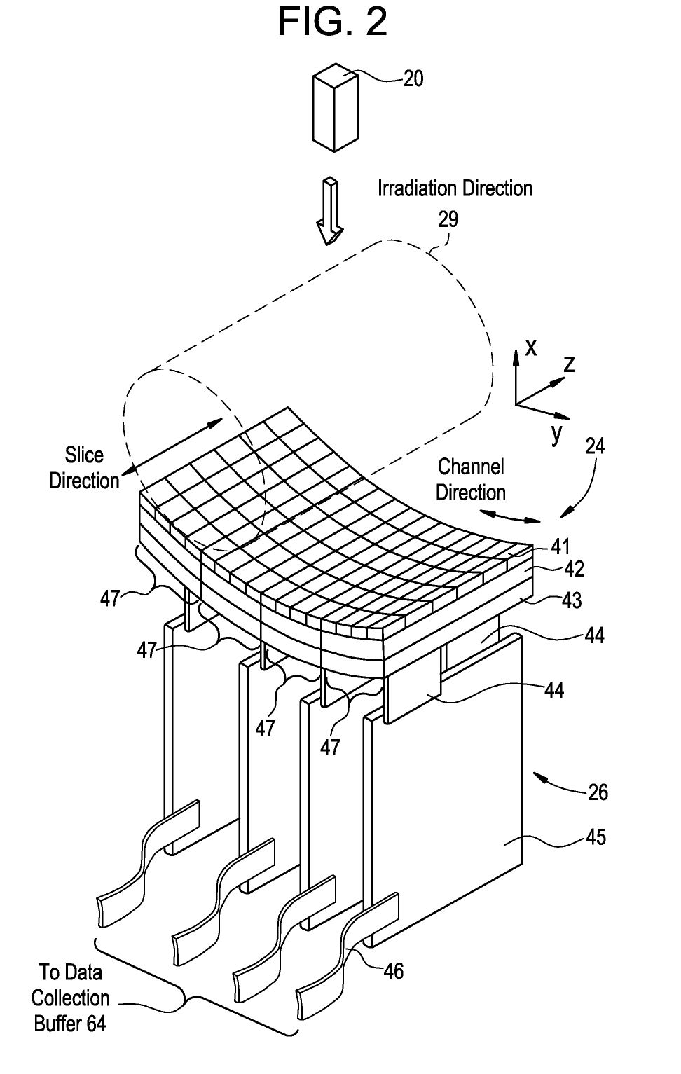

FIG. 2 is an outside drawing showing three-dimensional layout of the X-ray tube 20, the X-ray detector 24, and the data collector 26. The X-ray detector 24 includes scintillators 41 for detecting a conical X-ray beam generated by the X-ray tube 20, a photodiode 42 as a photoelectric converter for detecting light emission of the scintillators 41, a reflection film 48, and a plane board 43. Although the reflection film 48 exists on the two-dimensional array of the scintillators 41, it is not shown here.

The scintillators 41 are arranged two-dimensionally on the surface facing the conical X-ray beam and emit light when the X-ray enters. Approximately 64 scintillators 41 are arranged in the slice direction as the thickness direction of the conical X-ray beam and approximately 1,000 scintillators 41 are arranged in the channel direction as a spread direction of the fan shape of the X-ray beam.

The photodiode 42 is formed on the plane board 43 and detects light emission of the scintillators 41. On the plane board 43 as a single plane board, the scintillators 41 and the photodiodes 42 corresponding to a plurality of channels and a plurality of slices are formed. By the scintillators 41, the photodiodes 42, and the plane board 43 formed in an integral structure, a single plane block 47 is formed. By combination of a plurality of plane blocks 47, the X-ray detector 24 having an almost concave shape is constructed. In the example of FIG. 2, the plane blocks 47 of four channels and three slices are formed. The plane blocks 47 are arranged on a concave surface which is almost orthogonal to the incident conical X-ray beam.

The data collector 26 includes flexible printed boards 44, printed boards 45, and electric cables 46. The flexible printed board 44 transmits an analog signal of the X-ray detected by the photodiode 42 to the printed board 45.

The electric cable 46 is a flat cable electrically connected from an end in the slice direction to the printed board 45. The printed board 45 is electrically connected to the data collection buffer 64 via the electric cable 46.

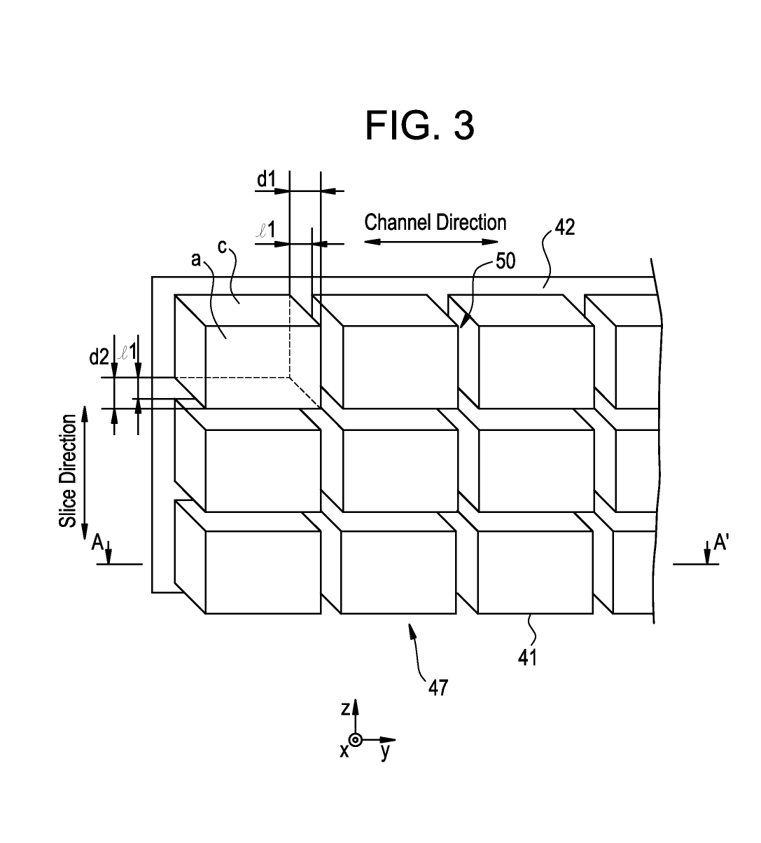

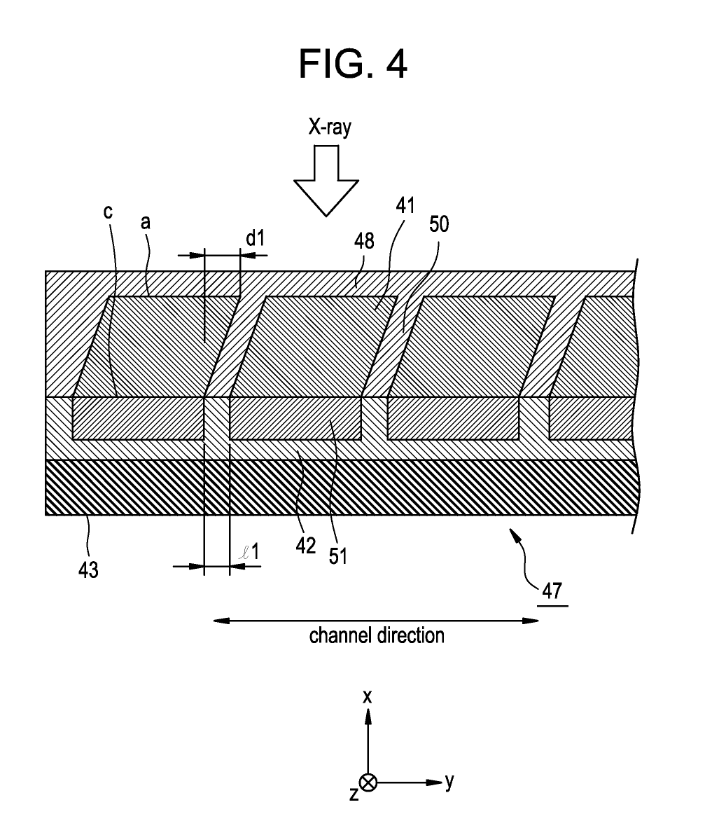

FIGS. 3 and 4 are diagrams showing the scintillators 41, the photodiode 42, and the plane board 43 constructing the plane block 47. In the following, the case where the plane block 47 is positioned in a YZ plane and the X-ray incidence direction is the X-axis direction will be described.

FIG. 3 is a plan view showing the plane block 47 viewed from the X axis direction as the X-ray incidence direction. Although the reflection film 48 which will be described later exists on the scintillators 41 of the plane block 47, it is not shown in FIG. 3 in order to clearly show the two-dimensional array of the scintillators 41. In FIG. 3, as an example, dot lines as hidden lines are shown only in the upper left scintillator 41.

Each of the scintillators 41 has a parallelepiped shape. The scintillators 41 having the same structure are repeatedly arranged two-dimensionally in the channel direction and the slice direction with gaps 50 therebetween. It is assumed here that the length of the gap 50 in the channel direction is l1, and the length of the gap 50 in the slice direction is l2.

A top face “a” orthogonal to the incident X-ray of the parallelepiped constructing the scintillator 41 and an under face “c” indicated by the dot lines in FIG. 3 are position-deviated from each other in the channel and slice directions. When the size of the positional deviation between the top face “a” and the under face “c” is d1 in the channel direction and is d2 in the slice direction, the following expressions are satisfied.

d1 >l1 in the channel direction

d2 >l2 in the slice direction

FIG. 4 is a cross section taken along line A-A′ when the scintillators 41 arranged two-dimensionally and shown in FIG. 3 are viewed from the z axis direction. On the scintillators 41 on the photodiode 42, the reflection film 48 which is not shown in FIG. 3 is illustrated. The reflection film 48 is made of a resin filler containing metal powders and is filled on the top of the scintillators 41 and in the gaps 50. FIG. 4 also shows an anode 51 of the photodiode 42. The anode 51 serves as a light receiving surface of the photodiode 42 and overlaps the under face “c” of the scintillator 41.

Scintillation light generated in the scintillator 41 by incidence of the X-ray is confined in the scintillator 41 by the reflection film 48 and detected by the anode 51. Leaked light among the scintillators 41 is also prevented by the reflection film 48 in the parts of the gaps 50.

As described above, the top face “a” is deviated from the under face “c” in the channel direction only by the amount d1. Since the amount is larger than the amount l1 of the gap 50 in the channel direction, when the plane block 47 is viewed from the X-ray incidence direction, the gap 50 cannot be seen except for the peripheral portions of the two-dimensional array.

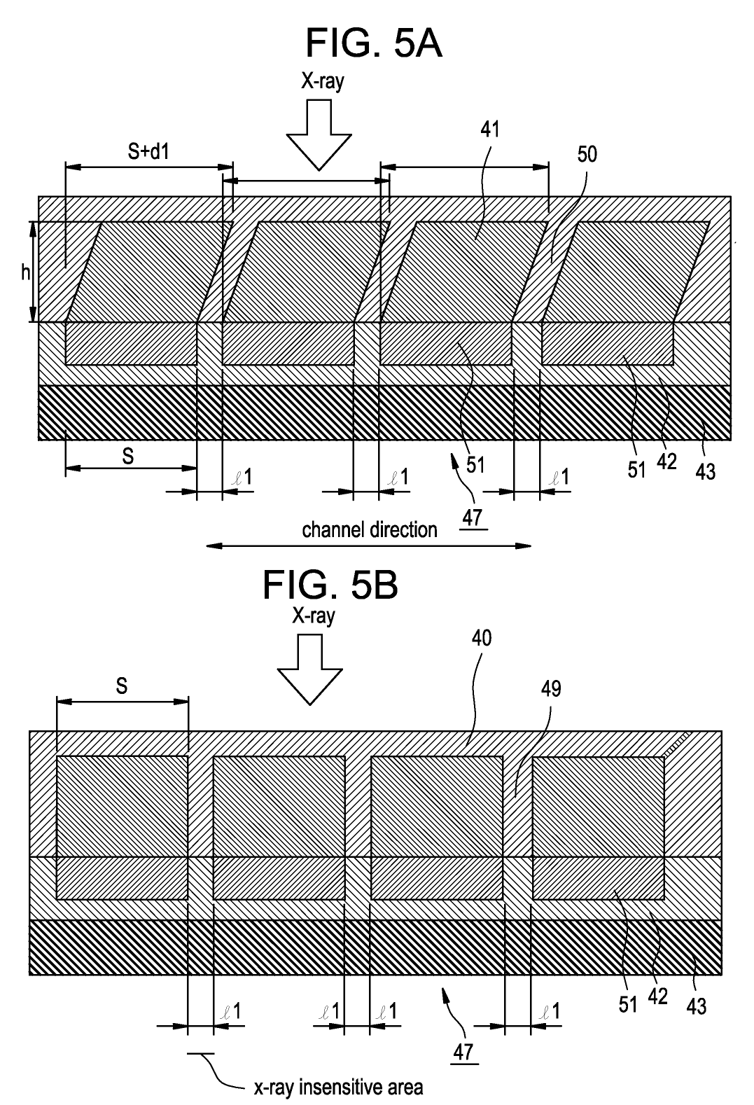

Next, the operation of the scintillators 41 according to the first embodiment will be described with reference to FIGS. 5A and 5B. FIG. 5A is an explanatory cross section taken along A-A′ line of FIG. 3 like FIG. 4. The scintillator 41 has a parallelepiped shape, and the top face “a” and the under face “c” are deviated from each other only by the amount d1 in the channel direction. Therefore, when it is assumed that the length in the channel direction of the top face “a” or the under face “c” is “s”, the length of an X-ray sensitive area in the channel direction of the scintillator 41 with respect to the X-ray entering from above is equal to s+d1. When the length s+d1 is compared with the length s+l1 (the length “s” in the channel direction of the under face “c” and the width l1 of the gap 50), the following expression is obtained.

s+d1 >s+l1

Therefore, the whole plane block 47 viewed from the X-ray incidence direction is covered with the X-ray sensitive areas of the scintillators 41, so that efficiency for X-ray utilization improves.

The X-ray sensitive areas of neighboring scintillators 41 overlap each other and hide the gap 50. Therefore, in end portions of the scintillators 41 under which the gap 50 exists, the scintillator length in the X-ray incident direction decreases, and the probability of absorbing incident X-rays decreases. In other words, the probability that the incident X-ray passes through the end portion of the scintillator 41 is high. To lessen the phenomenon, the height “h” in the X-ray incidence direction of the scintillator 41 is increased or the distance d1 of the deviation in the channel direction between the top face “a” and the under face “c” is increased, thereby narrowing the width of the gap 50 in the X-ray incidence direction or the like.

FIG. 5B is an explanatory diagram showing an example of the case where scintillators 40 each having a rectangular parallelepiped shape are arranged on the anode 51 for comparison with FIG. 5A. The length of the X-ray sensitive area in the channel direction of the scintillator 40 with respect to an X-ray entering from above is “s”. On the other hand, a gap 49 having a width l1 between the scintillators 40 is a complete X-ray insensitive area. Therefore, efficiency for X-ray utilization is about s/(s+l1) and is lower than that in the case of FIG. 5A.

Although the X-ray sensitive areas in the channel direction of the scintillators 41 are shown as an example in FIGS. 5A and 5B, there is similarly no X-ray insensitive area also in the slice direction, and efficiency for X-ray utilization improves.

As described above, in the first embodiment, the scintillator 41 has a parallelepiped structure in which the top face “a” and the under face “c” are deviated from each other in the channel and slice directions only by the amounts d1 and d2 exceeding the width in the orthogonal direction of the gap 50, thereby eliminating X-ray insensitive areas when viewed from the X-ray incidence direction. Thus, efficiency for X-ray utilization can be improved and, moreover, X-ray detectivity and the picture quality of a slice image captured can be improved.

### Second Embodiment

In the foregoing first embodiment, the scintillator 41 has a parallelepiped structure in which the top face “a” and the under face “c” are deviated from each other only by the amounts exceeding the width of the gap 50, thereby eliminating X-ray insensitive areas when viewed from the X-ray incidence direction, typified by the gaps 50. Alternately, by forming the scintillator in a rectangular parallelepiped structure and tilting the plane block on which the scintillators are mounted with respect to the incident X-ray, X-ray insensitive areas when viewed from the X-ray incidence direction can be eliminated. In a second embodiment, the case where the scintillator has a rectangular parallelepiped structure and the plane block is tilted with respect to the incident X-ray will be described. Since the general configuration of the invention according to the second embodiment is the same as that shown in FIG. 1, its detailed description will not be repeated here.

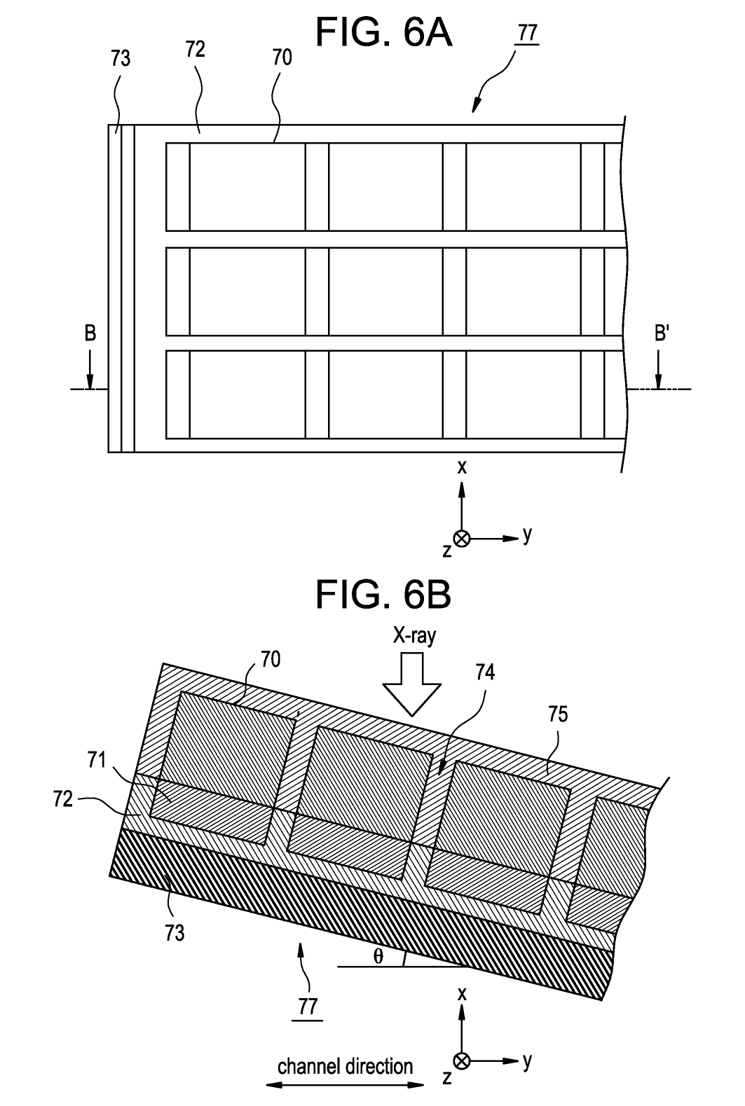

FIGS. 6A and 6B are diagrams showing the configuration of a plane block 77 according to the second embodiment. The plane block 77 corresponds to the plane block 47 including the scintillators 41, the photodiode 42, and the plane board 43 shown in FIG. 2. Since the other configuration is the same as that shown in FIG. 2, its detailed configuration will not be repeated.

The plane block 77 includes a reflection film 75, scintillators 70, a photodiode 72, and a plane board 73. Each of the scintillators 70 repeatedly arranged two-dimensionally in the channel direction and the slice direction has a rectangular parallelepiped shape, and emits light by incidence of an X-ray. The photodiode 72 converts the light emitted from the scintillator 70 into an electric signal when the scintillator 70 is mounted on an anode 71 as a photodetector. The scintillators 70 and the photodiode 72 are mounted on the plane board 73, and the plane board 73 is disposed at a predetermined tilt θ from the channel direction orthogonal to the incident X-ray.

FIG. 6A is a plan view showing the plane block 77 viewed from the X-axis direction as an X-ray incident direction. Although the reflection film 75 which will be described later exists on the scintillators 70 of the plane block 77, it is not shown in FIG. 6A so that the two-dimensional array of the scintillators 70 is clearly shown.

The scintillator 70 has a rectangular parallelepiped shape. The scintillators 70 having the same structure are repeatedly arranged with gaps 74 in the channel and slice directions.

FIG. 6B is a cross section taken along B-B′ line of the scintillators 70 arranged two-dimensionally and shown in FIG. 6A when viewed from the z-axis direction. The reflection film 75 which is not shown in FIG. 6A is shown on the scintillators 70 on the photodiode 72. Like the reflection film 48, the reflection film 75 confines scintillation light within the scintillator 70 and prevents leakage of light among the scintillators 70. The plane block 77, that is, the plane board 73 is tilted only by a predetermined tilt θ from the orthogonal direction orthogonal to the incident X-ray.

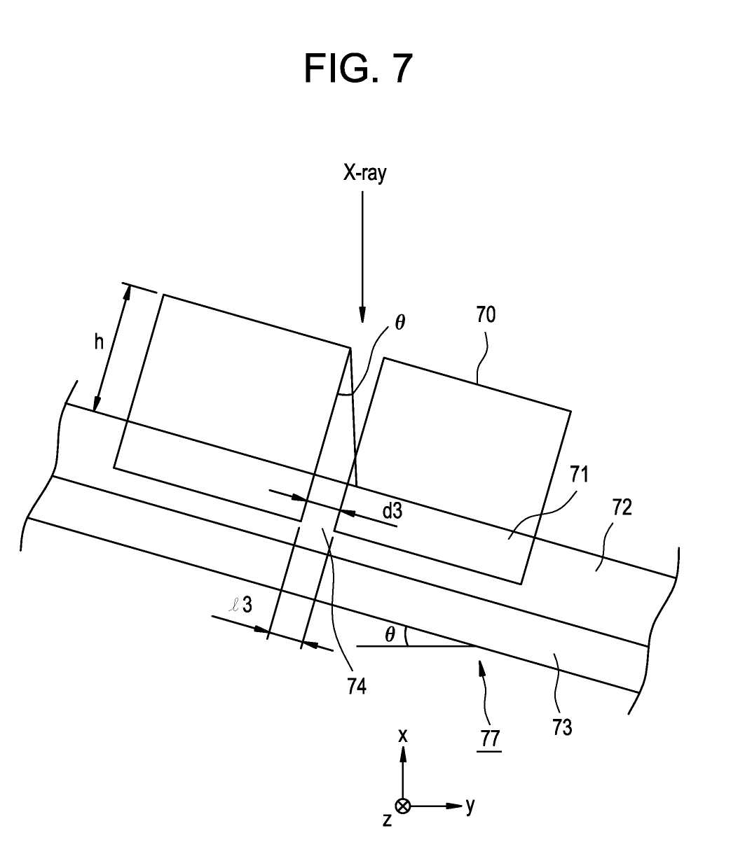

FIG. 7 is an explanatory diagram illustrating the magnitude of the tilt θ of the plane block 77. FIG. 7 is a cross section which is taken along line B-B′ shown in FIG. 6A in a manner similar to FIG. 6B. The height of the scintillator 70 having a rectangular parallelepiped from the photodiode 72 is set as “h” and the width of the gap 74 between the scintillators 70 is set as l3.

It is assumed that the shade of the scintillator 71 projected onto the photodiode 72 has a distance d3 from the end portion of the scintillator 71. In this case, the distance d3 is expressed as follows:
d3=h×tan(θ)

The tilt (θ) is set so that d3>l3, that is, h×tan(θ)>l3 is satisfied and no X-ray insensitive area exists when seen from the X-ray incidence direction.

As described above, in the second embodiment, the plane block 77 in which the scintillators 71 each having a rectangular parallelepiped shape are arranged two-dimensionally is tilted only by the tilt θ from the orthogonal direction which is orthogonal to the incident X-ray. Consequently, when viewed from the direction of the incident X-ray, there is no X-ray insensitive area due to the existence of the gaps 74 and the X-ray sensitive areas can be provided in almost the entire surface of the plane block 77. Thus, efficiency for X-ray utilization can be improved.

### Third Embodiment

In the first embodiment, the scintillator 41 has a parallelepiped structure in which the top face “a” and an under face “c” are deviated from each other by an amount exceeding the width of the gap 50, thereby eliminating the X-ray insensitive area when viewed from the X-ray incidence direction. Alternately, by using a multilayer scintillator as a multilayer solid state detector in which a plurality of scintillators each having a rectangular parallelepiped structure are stacked, similarly, an X-ray insensitive area of a two-dimensional scintillator array can be eliminated when viewed from the X-ray incidence direction. In the third embodiment, a multilayer scintillator in which a number of scintillators each having a rectangular parallelepiped structure are stacked will be disclosed. Since a general configuration of the invention according to the third embodiment is the same as that shown in FIG. 1, its detailed description will not be repeated.

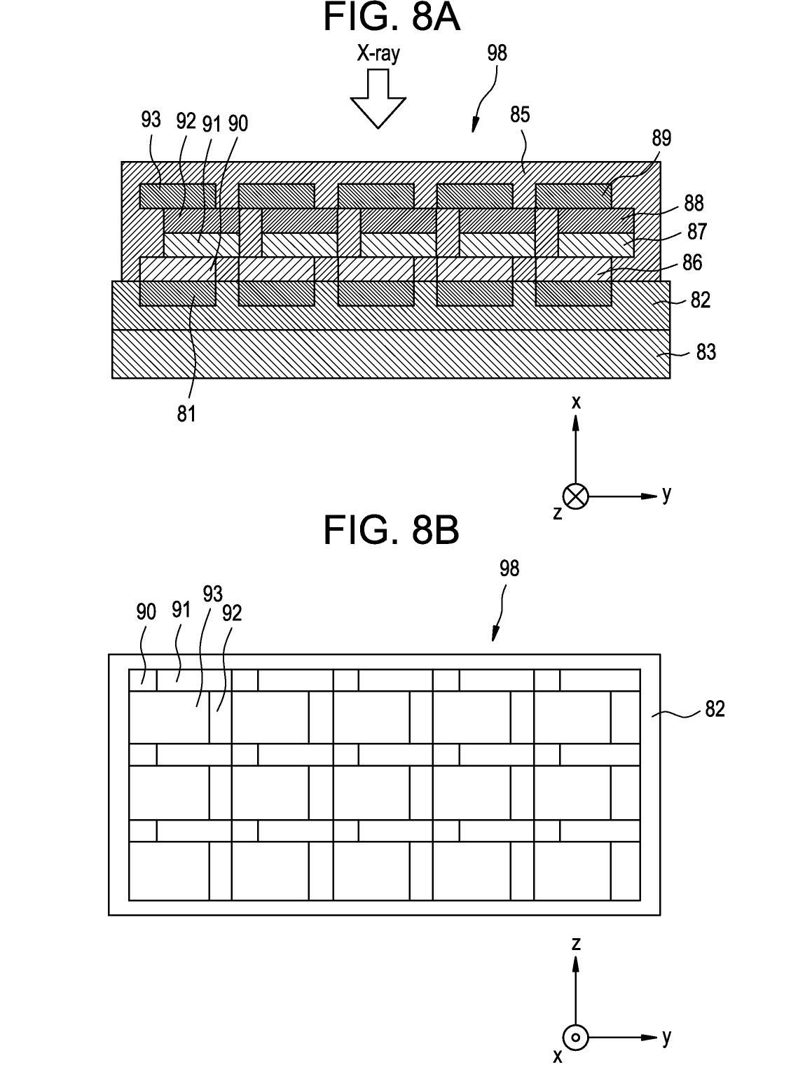

FIG. 8A is a cross section in the XY axis showing the configuration of a plane block 98 according to the third embodiment. The plane block 98 corresponds to the plane block 47 shown in FIG. 2, and the other configuration is the same as that shown in FIG. 2. The plane block 98 includes a reflection film 85, first to fourth layers 86 to 89 of multilayer scintillators, a photodiode 82, an anode 81, and a plane board 83. Since the reflection film 85, the photodiode 82, the anode 81, and the plane board 83 are the same as the reflection film 48, the photodiode 42, the anode 51, and the plane board 43 shown in FIG. 4, respectively, their description will not be repeated.

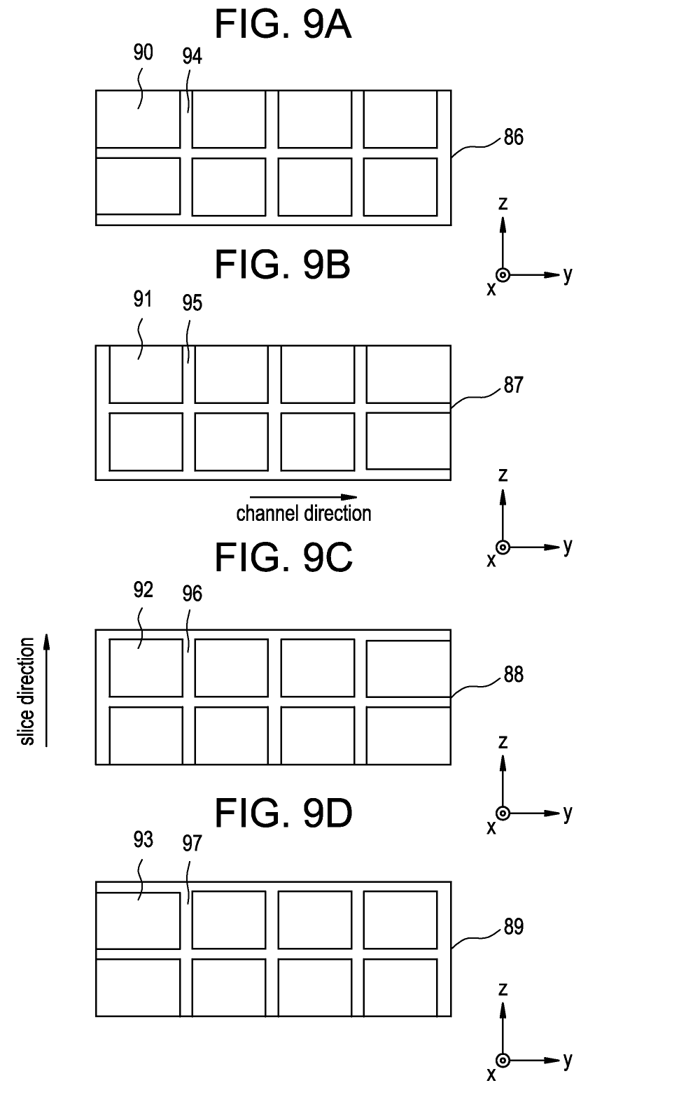

The first to fourth layers 86 to 89 of the multilayer scintillators form a multilayer solid state detector, and each of the scintillators has a rectangular parallelepiped shape. The first to fourth layers 86 to 89 are two-dimensionally-arranged four layers stacked in the X-ray incidence direction and whose relative positions are different from each other in the Y-axis or Z-axis direction. FIGS. 9A to 9D show the positions of the four multilayer scintillators from the X-axis direction as the X-ray incidence direction. The first to fourth layers 86 to 89 of FIGS. 9A to 9D are shown in a common frame, and the relative positions in the vertical and horizontal directions are shown.

FIG. 9A shows the first layer 86 from the X-axis direction as the X-ray incidence direction. The first layer 86 includes scintillators 90 each having a rectangular parallelepiped shape, which are arranged two-dimensionally, and gaps 94 between the scintillators. FIG. 9B shows the second layer 87 from the X-axis direction as the X-ray incidence direction. The second layer 87 includes scintillators 91 each having a rectangular parallelepiped shape, which are arranged two-dimensionally, and gaps 95 between the scintillators. The scintillator 91 has the same size as the scintillator 90, the gap 95 has the same width as the gap 94, and the scintillators 91 and the gaps 95 are moved only by the amount of the width of the gap 94 in the channel direction.

FIG. 9C shows the third layer 88 from the X-axis direction as the X-ray incidence direction. The third layer 88 includes scintillators 92 each having a rectangular parallelepiped shape, which are arranged two-dimensionally, and gaps 96 between the scintillators. The scintillator 92 has the same size as the scintillator 90, the gap 96 has the same width as the gap 94, and the scintillators 92 and the gaps 96 are moved only by the amount of the width of the gap 94 in the channel and slice directions. FIG. 9D shows the fourth layer 89 from the X-axis direction as the X-ray incidence direction. The fourth layer 89 includes scintillators 93 each having a rectangular parallelepiped shape, which are arranged two-dimensionally, and gaps 97 between the scintillators. The scintillator 93 has the same size as the scintillator 90, the gap 97 has the same width as the gap 94, and the scintillators 93 and the gaps 94 are moved only by the amount of the width of the gap 94 in the slice direction.

FIG. 8B is a diagram showing the plane block 98 in which the first to fourth layers 86 to 89 of the multilayer scintillators illustrated in FIGS. 9A to 9D are stacked, viewed from the X-ray incidence direction. The reflection film 85 covering the scintillators 90 to 93 is not shown in order to clearly show the positions of the first to fourth layers 86 to 89 viewed from the X-ray incidence direction.

When viewed from the X-ray incidence direction, the gaps 97 between the scintillators 93 (fourth layer 89) positioned in the uppermost layer in the X-ray incidence direction are covered with the scintillators 92 (third layer 88), the scintillators 91 (second layer 87), and the scintillators 90 (first layer 86) positioned in the lower layers. When viewed in the X-ray incidence direction, the X-ray insensitive areas in which there are no scintillators but the photodiode 82 is directly seen are only the peripheral parts of the two-dimensional array.

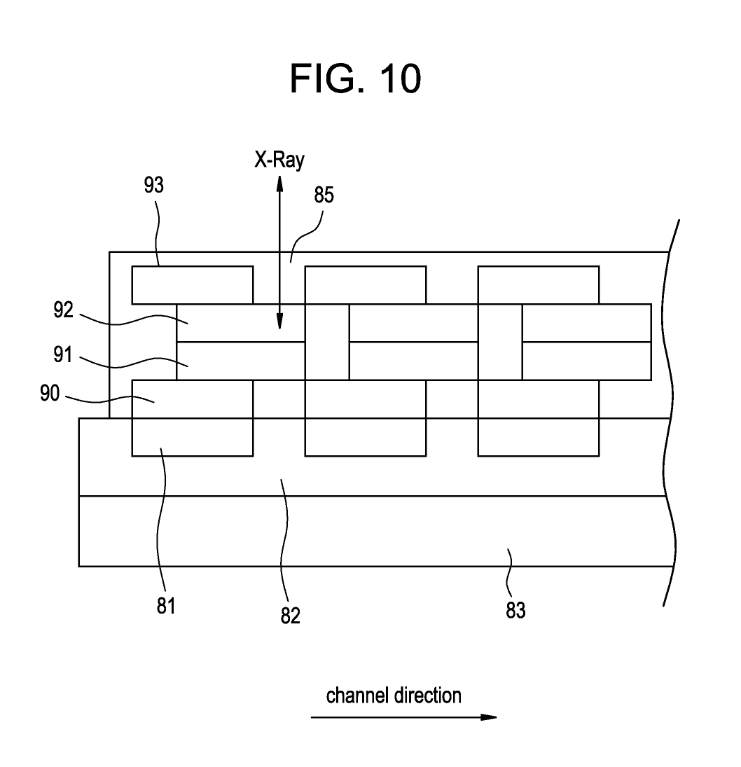

The operations performed by the first to fourth layers 86 to 89 of the multilayer scintillators when an X-ray is incident will be described with reference to FIG. 10. FIG. 10 is a section in the channel direction like FIG. 9A, showing, as an example, the case where an X-ray enters the gap 97 portion between the scintillators 93. The X-ray entering the gap 97 portion between the scintillators 93 is incident on at least one of the scintillators 92 and 91. By mutual action with one of the scintillators 92 and 91, fluorescence is generated. The fluorescence is multiple-reflected by the reflection film 85 surrounding the scintillators 90, 91, 92, and 93, finally absorbed by the anode 81, and converted to an electric signal. Although there is a portion partially in contact with the adjacent channel, since the contact portion is linear, it is considered that light leaked to the adjacent channel occurring in this portion is small.

The case where an X-ray enters the gap 96 portion between the scintillators 92, the case where an X-ray enters the gap 95 portion between the scintillators 91, and the case where an X-ray enters the gap 94 portion between the scintillators 90 are quite similar to the above case. Therefore, when the plane block 98 is viewed from the X-ray incidence direction, the X-ray insensitive areas exist only in the peripheries of the first to fourth layers 86 to 89 of the multilayer scintillators arranged two-dimensionally.

The thickness of the first to fourth layers 86 to 89 of the multilayer scintillators in the X-ray incidence direction is set to be optimum in consideration of the X-ray detection efficiency, weight, price, and the like. To be specific, since the scintillator itself of each layer is thin, the efficiency of detecting the X-rays entering the gaps 94 to 97 is low. Consequently, by increasing the detection efficiency by increasing the thickness in the X-ray incidence direction of each of the first to fourth layers 86 to 89 of the multilayer scintillators, the efficiency for X-ray utilization can be further increased.

As described above, in the third embodiment, the first to fourth layers 86 to 89 of the multilayer scintillators each having a rectangular parallelepiped shape are overlapped in a state where the relative positions of the layers are moved only by the widths of the gaps 94 to 97 in the channel direction and the slice direction, thereby eliminating the X-ray insensitive area in the plane block 98 when viewed from the X-ray incidence direction. Thus, an X-ray can be prevented from being undetected due to the gap between scintillators and, moreover, efficiency for X-ray utilization can be improved.

1. An X-ray detector in which a plurality of solid state detectors each having a parallelepiped shape are arranged in a two-dimensional array with gaps therebetween on a plane board facing an X-ray incidence direction,
wherein a relative position in the X-ray incidence direction of two parallel faces of each of the parallelepipeds have a positional deviation in the plane direction of the faces.

2. The X-ray detector according to claim 1, wherein the positional deviation is provided in at least one of a channel direction and a slice direction of the two-dimensional array.

3. The X-ray detector according to claim 1, wherein the positional deviation has a dimension exceeding width of the gap in the plane direction.

4. The X-ray detector according to claim 1, wherein the solid state detector is a scintillator.

5. The X-ray detector according to claim 4, wherein the plane board has a photodiode for detecting fluorescence generated by the scintillator.

6. An X-ray CT apparatus having an X-ray detector in which a plurality of solid state detectors each having a parallelepiped shape are arranged in a two-dimensional array with gaps therebetween on a plane board facing an X-ray incidence direction,
wherein a relative position in the X-ray incidence direction of two parallel faces of each of the parallelepipeds have a positional deviation in the plane direction of the faces.

7. The X-ray CT apparatus according to claim 6, wherein the positional deviation has a dimension exceeding width of the gap in the plane direction.

8. The X-ray CT apparatus according to claim 6, wherein the solid state detector is a scintillator.

9. The X-ray CT apparatus according to claim 8, wherein the plane board has a photodiode for detecting fluorescence generated by the scintillator.

10. An X-ray CT apparatus comprising:
an X-ray tube that generates an X-ray; and
an X-ray detector in which a plurality of solid state detectors each having a rectangular parallelepiped shape are arranged in a two-dimensional array with gaps therebetween on a plane board facing the X-ray incidence direction,
wherein the plane board being tilted with respect to a direction orthogonal to the incidence direction.

11. The X-ray CT apparatus according to claim 10, wherein the plane board being tilted so the X-ray projection of the rectangular parallelepiped as to exceed the gap and overlaps an adjacent rectangular parallelepiped.

12. The X-ray CT apparatus according to claim 10, wherein the solid state detector is a scintillator.

13. The X-ray CT apparatus according to claim 12, wherein the plane board has a photodiode for detecting fluorescence generated by the scintillator.

14. An X-ray detector in which a plurality of solid state detectors each having a rectangular parallelepiped shape are arranged in a two-dimensional array with gaps therebetween on a plane board facing an X-ray incidence direction,
wherein the X-ray detector has a multilayer solid state detector in which a plurality of the solid state detectors in the two-dimensional array are stacked in the incidence direction, and relative positions of the stacked solid-state detectors are deviated in a direction orthogonal to the stacked direction.

15. The X-ray detector according to claim 14, wherein the two-dimensional array has the relative position which varies in at least one of a channel direction and a slice direction as two arrangement directions of the two-dimensional array.

16. The X-ray detector according to claim 15, wherein the multilayer solid state detector has first, second, third, and fourth solid state detectors whose relative positions are different from each other.

17. The X-ray detector according to claims 14, wherein the solid state detector is a scintillator.

18. An X-ray CT apparatus comprising an X-ray detector in which a plurality of solid state detectors each having a rectangular parallelepiped shape are arranged in a two-dimensional array with gaps therebetween on a plane board facing an X-ray incidence direction,
wherein the X-ray detector has a multilayer solid state detector in which a plurality of the solid state detectors in the two-dimensional array are stacked in the incidence direction, and relative positions of the stacked solid-state detectors are deviated in a direction orthogonal to the stacked direction.

19. The X-ray CT apparatus according to claim 18, wherein in the multilayer solid state detector, the relative position varies in at least one of a channel direction and a slice direction as two arrangement directions of the two-dimensional array.

20. The X-ray CT apparatus according to claim 18, wherein the solid state detector is a scintillator.
